(19) United States
(12) Patent Application Publication
(10) Pub. No.: US 2010/0015704 A1
(43) Pub. Date: Jan. 21, 2010

(54) PROCESS FOR THE ENRICHMENT OF MELANOCYTES BY MEANS OF MODIFIED SURFACES

(75) Inventors: Heike Mertsching, Sindelfingen (DE); Petra Kluger, Gerlingen (DE); Herwig Brunner, Weilheim (DE); Christian Oehr, Herrenberg (DE); Vincenzo Sciarratta, Berlin (DE)

Correspondence Address: HARNESS, DICKEY & PIERCE, P.L.C., P.O. BOX 828, BLOOMFIELD HILLS, MI, 48303, US

(73) Assignee: FRAUNHOFER-GESELLSCHAFT ZUR FORDERUNG DER ANGEWAND, MUNCHEN (DE)

(21) Appl. No.: 12/447,596

(22) PCT Filed: Dec. 6, 2007
(86) PCT No.: PCT/EP07/10590

§ 371 Date: Apr. 28, 2009

(30) Foreign Application Priority Data
Dec. 7, 2006 (DE) 10 2006 057 742.6

(51) Int. Cl. C12N 5/08; C12N 5/00; C12M 3/00

(52) U.S. Cl. 435/350; 435/351; 435/371; 435/378; 435/289.1

(57) ABSTRACT

The present invention relates to processes for obtaining melanocytes from a cell suspension, in particular from an epidermal cell suspension, by means of a culture vessel, wherein at least a part of the surface of the culture vessel facing the culture space, is modified, in particular functionalized, in particular by means of a low-pressure plasma process. Furthermore, the present invention relates to culture vessels which are modified, in particular functionalized, in particular by means of a low-pressure plasma process and are suitable for obtaining melanocytes and the use of such culture vessels for obtaining melanocytes.

The present invention relates to processes for obtaining melanocytes from a cell suspension by means of a culture vessel, wherein at least a part of the culture vessel surface facing the culture space is modified, in particular functionalized. Furthermore, the present invention relates to culture vessels that are modified, in particular functionalized, by means of a low-pressure plasma process and are suitable for obtaining melanocytes, and the use of culture vessels of this type for obtaining melanocytes.

Approximately 90% of the human epidermis comprises keratinocytes. The other approximately 10% of the human epidermis is composed of dendritic cells, Langerhans' cells and pigment-forming melanocytes.

Melanocytes are cells capable of forming melanin. They occur in the skin in the basal layer, that is, the stratum basale, of the epidermis. The melanocytes thereby lie directly on the basilemma. Melanocytes are also found, for example, in the choroid and the iris of the eye, in the mucous membranes of the mouth and in the leptomeninges. Melanocytes are also found in the formation area of hair, in particular in the hair root.

Overall, obtaining cell cultures with a large proportion of melanocytes or pure melanocyte cultures is very complex.

Primary melanocytes are isolated above all from skin material, for example, human skin material. Depending on the donor material, the proportion of melanocytes is thereby less than 10%, in part less than 5% of the total cell count. A targeted isolation of this small cell population has hitherto been hardly possible. Selection methods such as FACS studies do not permit any further cultivation of the isolated melanocytes. The long-time cultivation of melanocytes is likewise difficult. Melanocytes usually exhibit in culture very poor proliferation rates, they differentiate quickly and develop tumor markers, so that the provision of grafts is rendered difficult in particular thereby. Compared to other cell types, melanocytes show high frequencies of DNA damage during the cultivation, which can lead to malignantly degenerate cells.

Tumorigenic changes of this type are a disadvantage not only with grafts, but also with the use of cells of this type in 3D test system for cosmetics, for example, for testing sunscreen agents and in the field of skin aging as an alternative method to animal testing.

In the prior art melanocytes are cultivated or obtained in conventional, unmodified culture vessels, in particular unmodified plastic vessels.

Cultivation methods are also known from the prior art in which the conventional, unmodified culture vessels are coated with gelatin, collagen or other protein compositions.

The technical problem of the present invention is the provision of a method for obtaining and/or isolating melanocytes, in particular a method that overcomes the aforementioned disadvantages at least in part.

Another technical problem on which the present invention is based is the provision of a method that renders possible the long-time cultivation of melanocytes.

The present invention solves the technical problem on which it is based through the provision of a method for obtaining melanocytes from a cell suspension, wherein the cell suspension is cultivated in a culture vessel at least for a period that is sufficient to ensure an adhesion of the melanocytes present in the cell suspension onto the surface of the culture vessel facing the culture space and wherein subsequently the adhering melanocytes are obtained, characterized in that at least a part of the surface of the culture vessel facing the culture space is modified.

Surprisingly, it has been shown that the method on which the invention is based makes it possible to obtain melanocytes, in particular pure melanocyte cultures, in a simple manner. Furthermore, it was surprisingly shown that the method according to the invention renders possible a long-time cultivation of melanocytes.

Surprisingly, it was shown that melanocytes from a primary epidermal cell suspension that contains less than 10% melanocytes, adhere to the surface of culture vessels modified, in particular functionalized by means of a plasma process, in particular a low-pressure plasma process or by means of a wet chemical process. The method according to the invention therefore makes it possible to provide melanocytes from a cell suspension, in particular an epidermal cell suspension, wherein in connection with the present invention the melanocytes thus obtained mean a melanocyte preparation that preferably contains at least 70% melanocytes. In a further preferred embodiment it is provided that the melanocyte preparation contains at least 80% melanocytes. In a further preferred embodiment it is provided that the melanocyte preparation contains at least 90% melanocytes. In a very particularly preferred embodiment it is provided that the melanocyte preparation is composed virtually solely or completely of melanocytes.

According to the invention a method for obtaining melanocytes from a cell suspension, in particular an epidermal cell suspension, wherein the cell suspension is cultivated in a culture vessel for a period that is sufficient to ensure an adhesion of the melanocytes present in the cell suspension to the surface of the culture vessel facing the culture space and wherein subsequently the adhering melanocytes are obtained, is thus characterized in that at least a part of the surface of the culture vessel facing the culture space is modified preferably by means of a plasma process, in particular a low-pressure plasma process.

In a particularly preferred embodiment it is provided that the modification of the surface part of the culture vessel facing the culture space is a functionalization with carboxyl groups. In a further preferred embodiment it is provided that the modification is carried out by means of a plasma process, in particular a low-pressure plasma process. In a further preferred embodiment it is provided that the modification is carried out by means of a wet chemical process. In a further preferred embodiment it is provided that the modifications, in particular the carboxyl groups applied, applied to the surface of the part of the culture vessel facing the culture space, are applied by means of a plasma process, in particular a low-pressure plasma process or by means of wet chemical methods. In a further preferred embodiment it is provided that the wet chemical methods provide, for example, the application of a polyelectrolyte, for example PAA (polyacrylic acid), preferably by means of the layer-by-layer technique with oppositely charged polyelectrolytes.

In a further preferred embodiment according to the invention it is provided that the at least one part of the surface of the culture vessel that is facing the culture space is roughened, in particular is present structured with microparticles and/or nanoparticles. In a particularly preferred embodiment a culture vessel is therefore used wherein at least a part of the surface of the culture vessel facing the culture space is modified, as described above, and furthermore has an increased roughness, in particular through applied nanostructures and/or microstructures, in particular microparticles and/or nanoparticles.

The invention therefore provides that in the process used according to the invention a culture vessel is used that is embodied such that at least a part of the surface of this culture vessel facing the culture space is modified, in particular functionalized, preferably by means of a plasma process, in particular a low-pressure plasma process and wherein a cell suspension, in particular an epidermal cell suspension is inserted into this culture vessel and cultivated so long and under such conditions that specifically and selectively the melanocytes present in the cell suspension adhere to the modified, in particular functionalized, surface of the culture vessel and in this manner can be separated. The separation of the adhering melanocytes is carried out by means of a manner known per se, for example, by decanting the cell suspension not adhering and washing and subsequent detaching of the adhering melanocytes.

Surprisingly, it was shown that melanocytes adhere very quickly to surfaces modified according to the invention. An adhesion of this type of the melanocytes to surfaces modified according to the invention could be observed after only ten minutes. Furthermore surprisingly it was shown that melanocytes adhere very particularly quickly to surfaces functionalized preferably by means of a plasma process, in particular low-pressure plasma process with carboxyl groups.

Preferably according to the invention the cell suspension is a cell suspension of a mammal, particularly preferably a human cell suspension. Preferably according to the invention the cell suspension is a human cell suspension. Preferably according to the invention the cell suspension is a murine cell suspension. Preferably according to the invention the cell suspension is a bovine cell suspension. Preferably according to the invention the cell suspension is a canine cell suspension. Preferably according to the invention the cell suspension is a porcine cell suspension. Preferably according to the invention the cell suspension is a feline cell suspension.

Preferably according to the invention the cell suspension is an epidermal cell suspension. Preferably according to the invention the epidermal cell suspension originates from an isolated skin section. Preferably according to the invention the cell suspension is an epidermal mammal cell suspension. Preferably according to the invention the cell suspension is a human epidermal cell suspension. Preferably according to the invention the epidermal cells of the cell suspension are cells from mouse, cow, rabbit, pig or cat. Preferably according to the invention the cell suspension is a murine, bovine, canine, porcine or feline epidermal cell suspension.

Preferably according to the invention, however, not only an epidermal cell suspension can be used, but also other cell suspensions that contain melanocytes. Examples of other sources for melanocytes are the choroid and the iris of the eye, the mucous membrane of the mouth or the leptomeninges. Preferably according to the invention the cell suspension comes from the choroid of the eye. Preferably according to the invention the cell suspension comes from the iris of the eye. Preferably according to the invention the cell suspension originates from the mucous membrane of the mouth. Preferably according to the invention the cell suspension originates from the leptomeninges. Preferably according to the invention the cell suspension, in particular the epidermal cell suspension, contains melanocytes from at least one hair root. Preferably according to the invention the cell suspension comprises melanocytes from at least one hair root.

In connection with the present invention “cell suspension” means a liquid cell culture, in particular at least one cell in liquid culture medium. According to the invention the cell suspension contains at least one melanocyte cell.

In a preferred embodiment the liquid culture medium can contain a base medium of amino acids, salt, trace elements and sugar as well as optionally additives, such as protein, antibiotics, growth factors, hormones, serum, etc., for example, additives such as calcium chloride, hFGF-B, PMA (tumor promoter) rh-insulin, hydrocortisone, BPE, FBS (serum) and/or gentamycin/amphotericin B. In particularly preferred embodiment a medium of this type can be used, wherein preferably no animal serum and/or no tumor promoters are used.

Preferably according to the invention the cell suspension is not directly added to a culture vessel modified according to the invention directly after isolation, but in an intermediate step first to a conventional, unmodified culture vessel intermediately cultivated. Preferably according to the invention before cultivation in the culture vessel the cell suspension is precultivated for 1 hour to 20 days in an unmodified culture vessel. Preferably according to the invention before the cultivation in the culture vessel the cell suspension is precultivated for 1 day to 15 days in an unmodified culture vessel. Preferably according to the invention before cultivation in the culture vessel the cell suspension is precultivated for 2 days to 10 days in an unmodified culture vessel. Likewise preferably according to the invention before cultivation in the culture vessel the cell suspension is precultivated for 1 minute to 60 minutes in an unmodified culture vessel. Likewise preferably according to the invention before cultivation in the culture vessel the cell suspension is precultivated for 10 minutes to 30 minutes in an unmodified culture vessel. However, it can also be preferred according to the invention that the cell suspension is not precultivated before the cultivation in the culture vessel. It is therefore also preferred according to the invention that the freshly isolated cell suspension is placed directly in a culture vessel modified according to the invention.

Preferably according to the invention a cell suspension is cultivated over a certain period in a culture vessel modified according to the invention. Preferably according to the invention the certain period is at least so long that an adhesion of at least a part, particularly preferably at least half, of the melanocytes contained in the cell suspension, in particular all of the melanocytes contained in the cell suspension is ensured. Preferably according to the invention the certain period is so long that an adhesion of at least a part of the melanocytes contained in the cell suspension is ensured.

According to the invention the certain period is so long that an adhesion of at least half of the melanocytes contained in the cell suspension is ensured. Preferably according to the invention the certain period is so long that an adhesion of all of the melanocytes contained in the cell suspension is ensured. However, the period can also be extended, in particular for storing the melanocytes. The cultivation duration can therefore be selected by one skilled in the art as required. While for simple enrichment of melanocytes a short period, in particular in the range of minutes and hours, can already be sufficient, cell suspensions can also be cultivated over a longer period, in particular over days and weeks, for preservation and storage of the melanocytes. One skilled in the art knows thereby whether and, if so, when the culture medium of the cell suspension is to be replaced or to be supplemented, for example nutrients and other additives for cultivation of the melanocytes have to be added to the culture medium.

Preferably according to the invention a cell suspension is cultivated in a culture vessel modified according to the invention for at least 5 minutes. Preferably according to the invention a cell suspension is cultivated in a culture vessel modified according to the invention for at least 10 minutes. Preferably according to the invention a cell suspension is cultivated in a culture vessel modified according to the invention for 30 minutes. Preferably according to the invention a cell suspension is cultivated in a culture vessel modified according to the invention for at least 1 hour. Preferably according to the invention a cell suspension is cultivated in a culture vessel modified according to the invention for at least 10 hours. Preferably according to the invention a cell suspension is cultivated in a culture vessel modified according to the invention for at least 1 day. Preferably according to the invention a cell suspension is cultivated in a culture vessel modified according to the invention for at least 5 days. Preferably according to the invention a cell suspension is cultivated in a culture vessel modified according to the invention for at least 6 days. Preferably according to the invention a cell suspension is cultivated in a culture vessel modified according to the invention for at least 1 week. Preferably according to the invention a cell suspension is cultivated in a culture vessel modified according to the invention for at least 2 weeks.

Preferably according to the invention a cell suspension is cultivated in a culture vessel modified according to the invention for at least 5 weeks.

Preferably according to the invention a cell suspension is cultivated in a culture vessel modified according to the invention for no more than 20 minutes. Preferably according to the invention a cell suspension is cultivated in a culture vessel modified according to the invention for no more than 30 minutes. Preferably according to the invention a cell suspension is cultivated in a culture vessel modified according to the invention for no more than 50 minutes. Preferably according to the invention a cell suspension is cultivated in a culture vessel modified according to the invention for no more than 1 hour. Preferably according to the invention a cell suspension is cultivated in a culture vessel modified according to the invention for no more than 2 hours. Preferably according to the invention a cell suspension is cultivated in a culture vessel modified according to the invention for no more than 15 hours. Preferably according to the invention a cell suspension is cultivated in a culture vessel modified according to the invention for no more than 1 day. Preferably according to the invention a cell suspension is cultivated in a culture vessel modified according to the invention for no more than 2 days. Preferably according to the invention a cell suspension is cultivated in a culture vessel modified according to the invention for no more than 7 days. Preferably according to the invention a cell suspension is cultivated in a culture vessel modified according to the invention for no more than 10 days. Preferably according to the invention a cell suspension is cultivated in a culture vessel modified according to the invention for no more than 2 weeks. Preferably according to the invention a cell suspension is cultivated in a culture vessel modified according to the invention for no more than 5 weeks. Preferably according to the invention a cell suspension is cultivated in a culture vessel modified according to the invention for no more than 7 weeks. Preferably according to the invention a cell suspension is cultivated in a culture vessel modified according to the invention for no more than 10 weeks. Preferably according to the invention a cell suspension is cultivated in a culture vessel modified according to the invention for no more than 1 month. Preferably according to the invention a cell suspension is cultivated in a culture vessel modified according to the invention for no more than 5 months. Preferably according to the invention a cell suspension is cultivated in a culture vessel modified according to the invention for no more than 7 months. Preferably according to the invention a cell suspension is cultivated in a culture vessel modified according to the invention for no more than 12 months.

Preferably according to the invention the cell suspension is cultivated for at least 10 minutes, preferably at least 20 minutes and no more than 2 hours, preferably no more than 1 hour.

According to the invention culture vessel is understood to mean a culture means that can be populated by cells, in particular melanocytes. Preferably according to the invention the culture vessel is a culture means that has a vessel form. Preferably according to the invention, however, the culture vessel can also have the structure of a carrier. Preferably according to the invention the carrier structure is a carrier structure of a graft.

Preferably according to the invention the culture vessel is a Petri dish. Preferably according to the invention the culture vessel is a microtitration plate. Preferably according to the invention the culture vessel is a multiwell plate. Preferably according to the invention the culture vessel is a culture flask

Preferably according to the invention the culture vessel is a culture vessel, particularly preferably a culture flask, in which a carrier structure of a graft is inserted in the culture space. Preferably according to the invention the carrier structure of the graft is a component of the culture vessel. Preferably according to the invention the carrier structure of the graft does not belong to the culture vessel. Preferably according to the invention the carrier structure of the graft is modified, in particular functionalized. Preferably according to the invention the carrier structure of the graft is not modified.

Preferably according to the invention the culture vessel is composed of plastic. Preferably according to the invention the surface of the culture vessel is composed of plastic. Preferably according to the invention the culture vessel is composed of at least one plastic. Preferably according to the invention the culture vessel contains plastic. Preferably according to the invention the surface of the culture vessel, in particular the surface facing the culture space, contains plastic. Preferably according to the invention the at least one plastic is a polymer plastic. Preferably according to the invention the at least one plastic is a standard plastic, in particular a plastic from the prior art.

Preferably according to the invention the plastic is selected from the group comprising polystyrene, polyethylene, polypropylene, polycarbonate, fluorinated polymers, polyvinyl chloride and mixtures thereof. Preferably according to the invention the surface of the culture vessel, in particular the surface facing the culture space, comprises at least one plastic, selected from the group comprising polystyrene, polyethylene, polypropylene, polycarbonate, fluorinated polymers, polyvinyl chloride and mixtures thereof. Preferably according to the invention the surface of the culture vessel, in particular the surface facing the culture space, comprises at least one plastic, selected from the group comprising polystyrene, polyethylene, polypropylene, polycarbonate, fluorinated polymers, polyvinyl chloride and mixtures thereof.

Preferably according to the invention the plastic is polystyrene. Preferably according to the invention the surface of the culture vessel, in particular the surface facing the culture space, contains polystyrene as plastic. Preferably according to the invention the surface, in particular the surface facing the culture space, of the culture vessel is composed of polystyrene. Preferably according to the invention the culture vessel is composed of polystyrene. Preferably according to the invention the plastic of the culture vessel is polystyrene.

Preferably according to the invention the plastic is polyethylene. Preferably according to the invention the surface of the culture vessel, in particular the surface facing the culture space, contains polyethylene as plastic. Preferably according to the invention the surface, in particular the surface facing the culture space, of the culture vessel is composed of polyethylene. Preferably according to the invention the culture vessel is composed of polyethylene. Preferably according to the invention the plastic of the culture vessel is polyethylene.

Preferably according to the invention the plastic is polypropylene. Preferably according to the invention the surface of the culture vessel, in particular the surface facing the culture space, contains polypropylene as plastic. Preferably according to the invention the surface, in particular the surface facing the culture space, of the culture vessel is composed of polypropylene. Preferably according to the invention the culture vessel is composed of polypropylene. Preferably according to the invention the plastic of the culture vessel is polypropylene.

In connection with the present invention a “culture vessel of plastic” means a vessel that is composed of plastic or contains plastic, wherein preferably the surface of the vessel is composed of plastic or contains plastic, and that is suitable to receive in particular to cultivate cell cultures, in particular cell suspensions.

Preferably according to the invention the culture vessel, in particular the surface of the culture vessel facing the culture space, can also comprise silicon. Preferably according to the invention the culture vessel, in particular the surface of the culture vessel facing the culture space, can also contain silicon. Preferably according to the invention the culture vessel, in particular the surface of the culture vessel facing the culture space, can also be composed of glass. Preferably according to the invention the culture vessel, in particular the surface of the culture vessel facing the culture space, can also contain glass. Preferably according to the invention the surface of the culture vessel, in particular the surface of the culture vessel facing the culture space, can contain at least one hydrogel. Preferably according to the invention the culture vessel, in particular the surface of the culture vessel facing the culture space, can be composed of at least one hydrogel.

In connection with the present invention the “surface of a culture vessel” means at least a part of the surface of the culture vessel, which as required comes into contact with the cell suspension and/or which is facing the culture space. The surface can be at least the base of the culture vessel. However, the surface can also be at least the inner side wall of the culture vessel. The surface can additionally, however, also include outer surfaces of the culture vessel, which do not come in contact with the cell suspension. Thus the surface of a culture vessel can also comprise all of the surfaces and walls of the culture vessel.

Preferably according to the invention the modified surface of the culture vessel is the entire surface of the culture vessel. Preferably according to the invention the modified surface of the culture vessel is the surface of the culture vessel facing the culture space. Preferably according to the invention the modified surface of the culture vessel is at least a part of the surface of the culture vessel facing the culture space. Preferably according to the invention the modified surface of the culture vessel is the base and/or the wall, in particular inner wall, of the culture space.

Culture space in connection with this invention means the part of a culture vessel which serves to receive at least one cell culture, in particular cell suspension. The culture space comprises according to the invention preferably at least one cavity. The cavity can have any form. Preferably according to the invention the cavity is formed by a base and one to four walls.

Preferably according to the invention at least a part of the surface of the culture vessel is functionalized. Preferably according to the invention at least part of the surface of the culture vessel is functionalized with carboxyl groups. Preferably according to the invention at least a part of the surface of the culture vessel facing the culture space is functionalized. Preferably according to the invention at least a part of the surface of the culture vessel facing the culture space is functionalized with carboxyl groups.

Preferably according to the invention the carboxyl groups are monofunctional carboxyl groups. Preferably according to the invention the carboxyl groups are generated by means of plasma polymerization, in particular by means of low-pressure plasma polymerization with acrylic acid on at least a part of the surface of the culture vessel, in particular on the surface facing the culture space.

Preferably according to the invention the modification density, particularly preferably functionalization density, is at least 0.1 carboxyl group per nm². Preferably according to the invention the modification density, particularly preferably functionalization density, is at least one carboxyl group per nm². Preferably according to the invention the modification density, particularly preferably functionalization density, is at least 2 carboxyl groups per nm². Preferably according to the invention the modification density, particularly preferably functionalization density, is at least 3 carboxyl groups per nm².

Preferably according to the invention the modification density, particularly preferably functionalization density, is no more than 200 carboxyl groups per nm². Preferably according to the invention the modification density, particularly preferably functionalization density, is no more than 100 carboxyl groups per nm². Preferably according to the invention the modification density, particularly preferably functionalization density is no more than 10 carboxyl groups per nm². Preferably according to the invention the modification density, particularly preferably functionalization density, is no more than 5 carboxyl groups per nm².

Preferably according to the invention the modification density, particularly preferably functionalization density, is at least 3 and no more than 5 carboxyl groups per nm². Preferably according to the invention the modification density, particularly preferably functionalization density is 4 carboxyl groups per nm².

In connection with the present invention, “functionalization” of a surface means a modification of the surface wherein functional chemical groups form or are applied on the surface. A functionalized surface is therefore a surface preferably modified according to the invention which has functional chemical groups, particularly preferably carboxyl groups.

Preferably according to the invention at least a part of the surface of the culture vessel facing the culture space is modified by means of a plasma process, in particular a low-pressure plasma process. Preferably according to the invention at least a part of the surface of the culture vessel facing the culture space is functionalized by means of a plasma process, in particular a low-pressure plasma process.

Preferably according to the invention with the plasma process, in particular low-pressure plasma process, in a first step the surface of the culture vessel is activated. Preferably according to the invention for the functionalization by means of the plasma process, in particular low-pressure plasma process, for activating the culture vessel, preferably the at least one part of the surface of the culture vessel facing toward the culture space at least one inert gas and/or at least one reactive gas is used. Preferably according to the invention an inert gas is used for the functionalization. The inert gas argon is preferred according to the invention. However, preferably according to the invention other inert gases and mixtures of different inert gases can also be used. Preferably according to the invention a reactive gas is used for the functionalization. Preferably according to the invention the reactive gas is selected from the group comprising oxygen, hydrogen, water or mixtures thereof. Preferably according to the invention the reactive gas is oxygen. Preferably according to the invention the reactive gas is hydrogen. Preferably according to the invention the reactive gas is water. Preferably according to the intention the reactive gas is a mixture of oxygen, hydrogen and water. However, preferably according to the invention other reactive gases and mixtures of different reactive gases can also be used.

Preferably according to the invention with the plasma process, in particular low-pressure plasma process, in a second step unsaturated finishing chemicals inserted into the gas phase are brought to reaction. Preferably according to the invention this results in stable surfaces with defined function densities depending on the deposition parameters. Preferably according to the invention the unsaturated finishing chemicals are ethylene oxide and/or acrylic acid. The unsaturated finishing chemicals are therefore used for modification, in particular functionalization. Preferably according to the invention ethylene oxide is used for the functionalization by means of the plasma process, in particular low-pressure plasma process. Preferably according to the invention acrylic acid is used for the functionalization by means of the plasma process, in particular low-pressure plasma process. Preferably according to the invention acrylic acid and/or ethylene oxide are used for the functionalization by means of the plasma process, in particular low-pressure plasma process. Particularly preferably acrylic acid is used for functionalization.

Preferably according to the invention the functionalization is carried out by means of a low-pressure plasma process at a pressure of 0.1 mbar to 1 mbar. Preferably according to the invention the functionalization is carried out by means of a low-pressure plasma process at a pressure of 0.3 mbar to 0.7 mbar. Preferably according to the invention the functionalization is carried out by means of a low-pressure plasma process at a pressure of 0.4 mbar to 0.6 mbar. Preferably according to the invention the functionalization is carried out by means of a low-pressure plasma process at a pressure of 0.5 mbar.

Preferably according to the invention the functionalization is carried out by means of a plasma process, in particular a low-pressure plasma process, over a period of 2 min to 60 min. Preferably according to the invention the functionalization is carried out by means of a plasma process, in particular a low-pressure plasma process, over a period of 5 min and 20 min. Preferably according to the invention the functionalization is carried out by means of a plasma process, in particular a low-pressure plasma process over a period of 10 min.

In addition to the plasma process, in particular low-pressure plasma process, preferably according to the invention other methods can also be used for modification, in particular functionalization, in particular wet chemical processes. For example, preferably according to the invention for modification, in particular functionalization the “soft lithography” method can be used in which functional groups, particularly preferably according to the invention carboxyl groups, are applied to the surface with a silicon stamp. Preferably according to the invention other printing techniques, for example to those of ink jet printers or spotters can also be used, as they are used for example in the field of microarray technology.

The invention also relates to a culture vessel for obtaining melanocytes, characterized in that at least a part of the surface of the culture vessel facing the culture space is modified, in particular functionalized. The culture vessel can be embodied as described above, in particular the culture vessel is a culture vessel, particularly preferably a culture flask, in which a carrier structure of a graft is inserted in the culture space.

Preferably according to the invention the culture vessel is composed of plastic. Preferably according to the invention the at least one part of the surface of the culture vessel facing the culture space is modified, in particular functionalized, by means of a plasma process, in particular a low-pressure plasma process. Preferably according to the invention at least a part of the surface of the culture vessel facing the culture space is functionalized with carboxyl groups. A culture vessel of the invention is particularly preferred wherein the at least one part of the surface of the culture vessel facing the culture space is structured with microparticles and/or nanoparticles.

The invention also includes the use of a culture vessel according to the invention for obtaining melanocytes, wherein at least a part of the surface of the culture vessel facing the culture space is modified, preferably functionalized, particularly preferably functionalized with carboxyl groups, preferably by means of a plasma process, in particular a low-pressure plasma process. The invention also includes the use of a culture vessel according to the invention for obtaining melanocytes, wherein at least a part of the surface of the culture vessel facing the culture space is modified. The invention also comprises the use of a culture vessel according to the invention for obtaining melanocytes wherein at least a part of the surface of the culture vessel facing the culture space is functionalized by means of a plasma process, in particular a low-pressure plasma process, or a wet chemical method with carboxyl groups.

### EXAMPLES

### Example 1

### Production of Modified Petri Dishes

Polystyrene Petri dishes were modified with carboxyl groups. The modification of the carrier material was carried out in a plasma chamber with continuous RF output at 20 W power. After the activation of the surfaces of the Petri dish for 4 seconds with hydrogen plasma, the treatment of the surfaces was carried out for 10 minutes with acrylic acid vapor at 0.5 mbar. A modification density of 4 carboxyl groups per nm² was produced. The carboxyl-modified Petri dishes were subsequently sterilized for 30 minutes in an ethanol bath and then dried.

### Example 2

### Obtaining Melanocytes

Epidermal cells precultivated for 5 to 10 days were detached from a conventional culture vessel by enzymatic digestion with trypsin. The cells obtained were taken up in culture medium. The cell suspension obtained was then applied to the carboxyl-modified surfaces of the Petri dishes. After only 10 to 20 minutes it was possible to recognize a clear enrichment of melanocytes on the carboxyl-modified surfaces.

1-32. (canceled)

33. A process for obtaining melanocytes from a cell suspension comprising: modifying at least a part of a surface of a culture vessel facing a culture space by functionalization with carboxyl groups;
cultivating the cell suspension in the culture vessel at least for a period that is sufficient to ensure an adhesion of the melanocytes present in the cell suspension to the surface of the culture vessel facing the culture space; and
subsequently obtaining the adhering melanocytes.

34. The method according to claim 33 comprising applying the carboxyl groups by plasma polymerization or by a wet chemical process.

35. The method according to claim 33, wherein the cell suspension is an epidermal cell suspension and/or a cell suspension of at least one hair root.

36. The method according to claim 33, wherein modifying at least the part of the surface of the culture vessel facing the culture space includes modifying by a plasma process.

37. The method according to claim 33, wherein functionalization includes functionalization with a low-pressure plasma process.

38. The method according to claim 33 comprising originating the cell suspension as an epidermal cell from an isolated skin section.

39. The method according to claim 33, wherein the cell suspension is an epidermal mammal cell suspension.

40. The method according to claim 33, wherein the cell suspension is a human epidermal cell suspension.

41. The method according to claim 33, wherein the cell suspension is a murine, bovine, canine, porcine or feline epidermal cell suspension.

42. The method according to claim 33, further comprising precultivating the cell suspension in the culture vessel for approximately 1 hour to approximately 20 days in an unmodified culture vessel before the cultivation.

43. The method according to claim 33, further comprising precultivating the cell suspension in the culture vessel for approximately 1 day to approximately 10 days before the cultivation.

44. The method according to claim 33, further comprising precultivating the cell suspension in the culture vessel for approximately 1 minute to approximately 60 minutes in an unmodified culture vessel before the cultivation.

45. The method according to claim 33, wherein the cell suspension is cultivated in the culture vessel for no more than approximately 2 hours.

46. The method according to claim 33, wherein the cell suspension is cultivated in the culture vessel for no more than approximately 1 hour.

47. The method according to claim 33, wherein the surface of the culture vessel contains plastic or is composed of plastic.

48. The method according to claim 33, wherein the surface of the culture vessel contains at least one plastic, selected from the group comprising polystyrene, polyethylene, polypropylene, polycarbonate, fluorinated polymers, polyvinyl chloride and mixtures thereof.

49. The method according to claim 33, wherein the culture vessel is composed of at least one plastic selected from the group comprising polystyrene, polyethylene, polypropylene, polycarbonate, fluorinated polymers, polyvinyl chloride and mixtures thereof.

50. The method according to claim 33, wherein the surface of the culture vessel contains silicon, glass, or a combination thereof.

51. The method according to claim 33, wherein the culture vessel is composed of silicon or glass.

52. The method according to claim 33, wherein the surface of the culture vessel contains at least one hydrogel.

53. The method according to claim 33, wherein the culture vessel is composed of at least one hydrogel.

54. The method according to claim 33, wherein the part of the surface of the culture vessel facing the culture space is functionalized by a low-pressure plasma process with carboxyl groups.

55. The method according to claim 34, wherein acrylic acid and/or ethylene oxide is used for the functionalization of the plasma process.

56. The method according to claim 34, further comprising using the functionalization by the plasma process, and using at least one gas selected from a group comprising inert gas, reactive gas, and combinations thereof to activate the at least one part of the surface of the culture vessel facing the culture space.

57. The method according to claim 33, comprising using a pressure of 0.1 mbar to 1 mbar for functionalization.

58. The method according to claim 33, comprising using a pressure of 0.3 mbar to 0.7 mbar for functionalization.

59. The method according to claim 33, comprising using a press of 0.5 mbar for functionalization.

60. The method according to claim 33, comprising carrying out the functionalization over a period of 2 minutes to 60 minutes.

61. The method according to claim 33, comprising carrying out the functionalization over a period of 5 minutes to 20 minutes.

62. The method according to claim 33, comprising carrying out the functionalization over a period of approximately 10 minutes.

63. The method according to claim 33, wherein the at least one part of the surface of the culture vessel facing the culture space is structured with microparticles and/or nanoparticles.

64. A culture vessel for obtaining melanocytes comprising: a surface having at least a part facing a culture space functionalized by a plasma process.

65. The culture vessel of claim 64, wherein the part of the surface is functionalized by a low-pressure plasma process or a wet chemical process.

66. The culture vessel according to claim 64, wherein the culture vessel is composed of plastic.

67. The culture vessel according to claim 64, wherein the culture vessel is functionalized with carboxyl groups.

68. The culture vessel according to claim 64, wherein the carrier structure is a carrier structure for a graft.

69. The culture vessel according to claim 64, wherein the at least one part of the surface of the culture vessel facing the culture space is structured with microparticles and/or nanoparticles.

70. A method of using of a culture vessel according to claim 64 to obtain a melanocyte.
